(19) United States
(12) Patent Application Publication
(10) Pub. No.: US 2018/0325155 A1
(43) Pub. Date: Nov. 15, 2018

(54) Method for Eliminating Harmful Substances from Plant Extracts

(71) Applicant: Hunan Nuoz Biological Technology Co., Ltd., Yiyang City (CN)

(72) Inventors: Wei Chen, Yiyang (CN); Zhimou Liu, Yiyang (CN)

(73) Assignee: Hunan Nuoz Biological Technology Co., Ltd., Yiyang City (CN)

(21) Appl. No.: 15/595,553

(22) Filed: May 15, 2017

(51) Int. Cl. A23L 33/105; C07H 1/08; A61K 31/00; A61K 45/06

(52) U.S. Cl. CPC A23L 33/105; C07H 1/08; A61K 36/074; A61K 45/06; A61K 31/00

(57) ABSTRACT

The present invention discloses a method for eliminating harmful substances from plant extracts. The raw materials for said plant extracts includes schisandra chinensis, rhodiola rosea L., ginseng root, ginseng steam and leaf, and etc.; the method comprises the following steps: extracting the pre-treated plant raw materials intended to be extracted, adding absorbent for absorbing harmful materials to the extract solution of the plant raw materials or to the re-dissolved solution of the plant extracts, and then being heated and stirred, the solids obtained from drying the suction filtrate are the plant extracts that have eliminated harmful substances therein. The present invention can deal with the excessive polycyclic aromatic hydrocarbons and Arsenic residues, once and for all, in a steady, effective, safe and controllable, low-cost, and low-pollution manner; in addition, the products of the present invention have a high recovery rate.

### FIELD OF THE INVENTION

The present invention is directed to the field of Chinese medicine preparation, and specifically to a method for eliminating harmful substances from plant extracts.

### BACKGROUND OF THE INVENTION

At present, many of extracts, obtained by conventional plant extraction methods, have a serious disadvantage of excessive polycyclic aromatic hydrocarbons and heavy metals arsenic residues, which is difficult to meet the latest standards of export to Europe and the United States, and constrains the promotion of Chinese medicine extracts, including schisandra chinensis, rhodiola rosea L., ginseng root, ginseng steam and leaf, Ganoderma Lucidum Karst, and etc., in oversea markets.

### SUMMARY OF THE INVENTION

It is an object of the present invention to provide a method for eliminating harmful substances from plant extracts, intended to solve the problem of seriously excessive polycyclic aromatic hydrocarbons and heavy metals arsenic residues in the extracts obtained by the conventional plant extraction methods, so as to meet or even exceed the latest standards of export to Europe and the United States.

The present invention is achieved in such a way that, a method for eliminating harmful substances from plant extracts, wherein raw materials of said plant extracts include schisandra chinensis, rhodiola rosea L., ginseng root, ginseng steam and leaf; the method comprises the following steps:

adding absorbent for absorbing harmful substances to an extract solution of the plant extracts or to a re-dissolved solution of plant extract solids, and then being heated to 50° C.-80° C., and stirred for 0.4 h-2.0 h, solids obtained from drying suction filtrate are the plant extracts that the harmful substances therein have been eliminated, wherein said harmful substances include polycyclic aromatic hydrocarbons, heavy metals including arsenic; wherein said absorbent comprises activated carbon powder with 100-300 meshes, an adding amount of said activated carbon powder corresponds to 0.05%-1.0%, on mass basis, of a solvent mass within the extract solution of the plant extracts or within the re-dissolved solution of plant extract solids; said solvent is ethanol, water, or a mixed solvent of ethanol and water.

Preferably, the extract solution of the plant extracts or the re-dissolved solution of plant extract solids is heated to 60° C.-80° C., and stirred for 0.5 h-1.0 h.

Preferably, said activated carbon powder has 200-300 meshes.

Preferably, the adding amount of the activated carbon powder corresponds to 0.05%-0.8%, on mass basis, of a solvent mass within the extract solution of the plant extracts or within the re-dissolved solution of plant extract solids.

Preferably, the adding amount of the activated carbon powder corresponds to 0.05%-0.6%, on mass basis, of a solvent mass within the extract solution of the plant extracts or within the re-dissolved solution of plant extract solids.

Preferably, said absorbent further comprises magnesium oxide adsorbent of which the adding amount corresponds to 0.5%-4.0%, on mass basis, of a solvent mass within the extract solution of the plant extracts or within the re-dissolved solution of plant extract solids.

Compared with the prior art, the present invention has the following beneficial effects: dealing with the excessive polycyclic aromatic hydrocarbons and arsenic residues, once and for all, in a steady, effective, safe and controllable, low-cost and low-pollution manner, and thereby achieving the plant extract products meeting the latest standards of export to Europe and United States; in addition, the products of the present invention have a high recovery rate.

### DETAILED EMBODIMENT OF THE INVENTION

The present invention will be described in further detail with reference to the following embodiments, for the purpose of making the objectives, technical solutions and advantages of the present invention clearer. It shall be understood that the specific embodiments described herein are illustrative only for the invention and not intended to limit the invention.

In the following embodiments, all the activated carbon powders are purchased from Durban Activated Carbon Co., Ltd., Model 200-11.

### Embodiment 1

Taking 150.00 g of schisandra chinensis extract dry powder (total 11.74%) with excessive polycyclic aromatic hydrocarbons and heavy metals arsenic residues, adding 75% ethanol with 5 times the mass of the dry powder to dissolve the dry powder, and adding 0.75 g of activated carbon powder with 200-300 meshes corresponding to 0.1% of the solvent mass, and 7.50 g of oxidation magnesium adsorbent corresponding to 1.0% of the solvent mass, and being stirred at 60° C. for 1.0 h afterward, and then being suction filtrated, till the filtrate being concentrated to be free of ethanol taste, and adding 80.00 g of β-cyclodextrin and grinded evenly, afterwards vacuum drying below 65° C., schisandra chinensis extract 1 is obtained, and then submitted for inspecting content, arsenic residues and to SINO/EUROFINS for inspecting PAH4 residues.

### Embodiment 2

Taking 150.00 g of schisandra chinensis extract dry powder (total 11.74%) with excessive polycyclic aromatic hydrocarbons and heavy metals arsenic residues, adding 75% ethanol with 8 times the mass of the dry powder to dissolve the dry powder, and adding 1.20 g of activated carbon powder with 200-300 meshes corresponding to 0.1% of the solvent mass, and 12.01 g of oxidation magnesium adsorbent corresponding to 1.0% of the solvent mass, and being stirred at 70° C. for 0.5 h afterward, and then being suction filtrated, till the filtrate being concentrated to be free of ethanol taste, and adding 80.00 g of β-cyclodextrin and grinded evenly, and afterwards vacuum drying below 65° C., schisandra chinensis extract 2 is obtained, and then submitted for inspecting content, arsenic residues and to Sino/EUROFINS for inspecting PAH4 residues.

### Embodiment 3

Taking 150.00 g of schisandra chinensis extract dry powder (total 11.74%) with excessive polycyclic aromatic hydrocarbons and heavy metals arsenic residues, adding 75% ethanol with 8 times the mass of the dry powder to dissolve the dry powder, and adding 11.99 g of activated carbon powder with 200-300 meshes corresponding to 1.0% of the solvent mass, and 36.01 g of oxidation magnesium adsorbent corresponding to 3.0% of the solvent mass, and being stirred at 50° C. for 2.0 h afterward, and then being suction filtrated, till the filtrate being concentrated to be free of ethanol taste, and adding 80.00 g of β-cyclodextrin and grinded evenly, and afterwards vacuum drying below 65° C., schisandra chinensis extract 3 is obtained, and then submitted for inspecting content, arsenic residues and to Sino/EUROFINS for inspecting PAH4 residues.

### Effect of Embodiments

Analyzing the Yield, content, and Benzo Pyrene residues obtained from the embodiments above.

1. Schisandra chinensis (yield of effective ingredient) (total of schisandra chinensis raw materials is 11.74%)

| | | | | | | Pseudo-r- | | | |
|---|---|---|---|---|---|---|---|---|---|
| Serial number | Schisandrin A | Schisandrol B | Schisantherin A | Deoxyschizandrin | γ-Schizandrin | Schizandrin | Total | Mass | Yield |
| Schisandra | 6.54% | 1.73% | 0.53% | 1.86% | 3.02% | 0.37% | 14.05% | 124.00 g | 98.9% |
| chinensis | | | | | | | | | |
| extract 1 | | | | | | | | | |
| Schisandra | 6.49% | 1.76% | 0.51% | 1.78% | 2.97% | 0.36% | 13.87% | 125.31 g | 98.7% |
| chinensis | | | | | | | | | |
| extract 2 | | | | | | | | | |
| Schisandra | 6.53% | 1.74% | 0.62% | 1.88% | 3.14% | 0.37% | 14.28% | 120.61 g | 97.8% |
| chinensis | | | | | | | | | |
| extract 3 | | | | | | | | | |

In the table above, the schisandra chinensis extracts obtained from embodiments 1˜3 are designated with the label of 1˜3; and as can be seen from the table above, the yield of the method of the present invention is high.

2. Analyses of residual quantity of harmful substances (European standard: PAH4<50 μg/kg, Benzo Pyrene<10 μg/kg, American Pharmacopoeia: Arsenic<2 mg/kg)

| | Benzo (a) | Benzo (a) | Benzo (b) | | | |
|---|---|---|---|---|---|---|
| | Anthracene | Pyrene | Fluoranthene | Chrysene | PAH4 | Arsenic |
| Serial number | μg/kg | μg/kg | μg/kg | μg/kg | μg/kg | mg/kg |
| Raw materials | 140 | 95 | 130 | 210 | 575 | 9.68 |
| of schisandra | | | | | | |
| chinensis | | | | | | |
| extract | | | | | | |
| Schisandra | 2.4 | 1.3 | 0.7 | 2.9 | 7.3 | 0.261 |
| chinensis | | | | | | |
| extract 1 | | | | | | |
| Schisandra | 2.3 | 1.1 | 0.6 | 3.0 | 7.0 | 0.200 |
| chinensis | | | | | | |
| extract 2 | | | | | | |
| Schisandra | 2.1 | 0.9 | 0.8 | 2.6 | 6.4 | 0.098 |
| chinensis | | | | | | |
| extracts 3 | | | | | | |

From the above analyses, it can be seen that the present invention utilizes a combined adsorbent comprising set proportions of magnesium oxide and activated carbon, which is to be heated and stirred in a solution with water and alcohol as solvent, and then suction filtrated, concentrated, dried, dealing with the excessive polycyclic aromatic hydrocarbons and arsenic residues, once and for all, and thereby achieving the plant extract products meeting the latest standards of export to Europe and United States.

In the present invention, a combined adsorbent comprising magnesium oxide and activated carbon deals with the excessive polycyclic aromatic hydrocarbons and arsenic residues, once and for all, in a steady, effective, safe and controllable, low-cost and low-pollution manner.

The above-described embodiments are preferred embodiments of the present invention, and not intended to limit the invention, any modifications, equivalent substitutions and improvements within the spirit and principles of the invention shall be included within the scope of the present invention.

What is claimed is:

1. A method for eliminating harmful substances from plant extracts, characterized in that the raw materials of the said plant extracts includes schisandra chinensis, rhodiola rosea L., ginseng root, ginseng steam and leaf; the method comprises the following steps:
adding absorbent for absorbing harmful substances to an extract solution of the plant extracts or to a re-dissolved solution of plant extract solids, and then being heated to 50° C.−80° C., and stirred for 0.4 h-2.0 h, solids obtained from drying suction filtrate are the plant extracts that the harmful substances therein have been eliminated, wherein said harmful substances include polycyclic aromatic hydrocarbons, heavy metals including arsenic; wherein said absorbent comprises activated carbon powder with 100-300 meshes, an adding amount of said activated carbon powder corresponds to 0.05%˜1.0%, on mass basis, of a solvent mass within the extract solution of the plant extracts or within the re-dissolved solution of plant extract solids;
said solvent is ethanol, water, or a mixed solvent of ethanol and water.

2. A method according to claim 1, characterized in that the extract solution of the plant extracts or the re-dissolved solution of plant extract solids is heated to 60° C.-80° C., and stirred for 0.5 h-1.0 h.

3. A method according to claim 1, characterized in that said activated carbon powder has 200˜300 meshes.

4. A method according to claim 1, characterized in that the adding amount of the activated carbon powder corresponds to 0.05%-0.8%, on mass basis, of a solvent mass within the extract solution of the plant extracts or within the re-dissolved solution of plant extract solids.

5. A method according to claim 1, characterized in that the adding amount of the activated carbon powder corresponds to 0.05%-0.6%, on mass basis, of a solvent mass within the extract solution of the plant extracts or within the s re-dissolved solution of plant extract solids.

6. A method according to claim 1, characterized in that said absorbent further comprises magnesium oxide adsorbent of which the adding amount corresponds to 0.5%-4.0%, on mass basis, of a solvent mass within the extract solution of the plant extracts or within the re-dissolved solution of plant extract solids.
